(19) United States
(12) Patent Application Publication
(10) Pub. No.: US 2012/0203099 A1
(43) Pub. Date: Aug. 9, 2012

(54) METHOD AND APPARATUS FOR LOCALIZATION OF INTRODUCED OBJECTS IN INTERVENTIONAL MAGNETIC RESONANCE

(76) Inventors: Andre De Oliveira, Erlangen (DE); Stephan Kannengiesser, Wuppertal (DE); Martin Requardt, Nuernberg (DE)

(21) Appl. No.: 13/369,608

(22) Filed: Feb. 9, 2012

(30) Foreign Application Priority Data
Feb. 9, 2011 (DE) 10 2011 003 874.4

(51) Int. Cl. A61B 5/055

(52) U.S. Cl. 600/414

(57) ABSTRACT

In a method and a magnetic resonance system to show an object that is introduced into an examination region, the object having a known chemical shift relative to tissue that is predominant in the examination region, magnetic resonance signals are acquired from the examination region of the subject with the introduced object therein, and the different chemical shift of the introduced object and of the predominant tissue is computationally used in a processor to calculate, from the acquired magnetic resonance signals, a localization image in which substantially only the introduced object is shown.

### BACKGROUND OF THE INVENTION

1. Field of the Invention

The present invention concerns a method to show, in a magnetic resonance (MR) image, an object introduced into an examination region, the object having a known chemical shift relative to the tissue that is predominant in the examination region, and a magnetic resonance system for implementing such a method.

2. Description of the Prior Art

In interventional applications supervised by MR, the MR images generated by a magnetic resonance system are used in order to localize the objects (such as catheter, laser or biopsy needle, for example) introduced in the intervention. Active localization methods and passive localization methods are known for this purpose. In active methods, micro-coils are used that are attached to the introduced subject (the catheter or a biopsy needle, for example). The MR signals induced in the imaging of the micro-coils can be detected and shown in the MR image. This technique, however, entails the risk that the micro-coils introduced into the body will be heated during the imaging. The SAR (Specific Absorption Rate) value, which indicates how much supplied heat is tolerable per volume or weight in the data acquisition, could hereby be exceeded. See among other things Nitz R W et al: On the Heating of Linear Conductive Structures as Guide Wires and Catheters in Interventional MRI, JMRI, 13:105-114 (2001) and Bock M. et al., MR-Guided Intravascular Interventions: Techniques and Applications, JMRI 27:326-338 (2008). Due to the danger of the increased heat absorption, these active localization methods have not become accepted in practice.

In addition to active localization methods, methods known as passive localization methods are known. These are based on the fact that prior knowledge about the shape of the object to be detected is known. In this case, image processing algorithms are used that determine predefined features in the acquired MR images that depend on the shape of the introduced object. One example of this passive tracking in the images via post-processing is described in de Oliveira A et al.: Automatic Passive Tracking of an Endorectal Prostate Biopsy Device Using Phase-Only Cross-Correlation, MRM 59:1043-1050 (2008), or in Busse H., et al.: Flexible Add-on Solution for MR Image-Guided Interventions in a Closed-Bore Scanner Environment, MRM 64:922-928 (2010). One disadvantage of this method is that the feature extraction from the generated MR image functions only to a satisfactory extent when no objects that have a shape similar to that of the introduced object are present in the examined region. An additional disadvantage exists in that additional slices must be acquired in order to determine the position of the introduced object, which excessively increases the MR examination time of an examined person. An additional passive method is described in Bock M. et al: A Faraday effect position sensor for interventional magnetic imaging, Phys Med Biol, 51(4):993-999 (2006). Due to the complexity of the method, this method has likewise not become established in practice.

### SUMMARY OF THE INVENTION

An object of the present invention is to provide a reliable detection, in an MR image, of an object introduced in an examination region.

According to the invention, a method is provided to depict an object introduced into an examination region, which object has a known MR chemical shift relative to the tissue predominant in the examination region. According to the invention, the different chemical shift of the introduced object and of the predominant tissue is used in order to calculate (with the use of acquired MR signals) a localization image in which essentially only the introduced object is depicted. This method belongs among the methods of passive tracking and utilizes the different chemical shift for extraction of the signals of the introduced object. The examining physician who has introduced the object into the examination region receives important information about the location of the introduced object.

In a preferred embodiment, the introduced object depicted in the localization image can be combined with an additional MR image acquired by the MR system. By the presentation of the introduced object in this additional MR image, the examining physician receives important information about the position of the introduced object in the examination region. For example, the additional image can be a phase image of the examination region. By calculating the localization image, it is possible to determine a position of the introduced object in the examination region. This position can then be used to depict the object.

One possibility to create the localization image is to acquire first MR signals with a gradient echo imaging sequence such that a magnetization of the predominant tissue in the examination region and the magnetization of the introduced object have essentially the same phase position at the echo point in time of the gradient echo. Furthermore, second MR signals can be acquired with a gradient echo imaging sequence in which the magnetization of the predominant tissue in the examination region and the magnetization of the introduced object have essentially the opposite phase position at the echo point in time. On the basis of the first and second MR signals it is possible to calculate the localization image in which the signal intensity corresponds to a proportion of the introduced object in the total signal. Finally, the localization image can be shown. These first and second MR signals can be generated with two different echo points in time in a single gradient echo imaging sequence or via two different acquisitions. In the first case, after radiating an RF pulse both the first signal and the second MR signal echo are acquired, while in the second case only the first or the second MR signal is respectively read out after radiation of an RF pulse to excite the magnetization. The first example is also known under the name “double echo sequence”.

One possibility to calculate the localization image in which the signal intensity corresponds to the proportion of the introduced object in the total signal is to generate the localization image using only image points to form the localization image in which a signal proportion of the introduced object in the total signal is greater than a predefined limit value. For example, only image points in which the signal proportion of the introduced object is greater than a predetermined percentile contribute to the localization image. For example, only image points in which the introduced object has a proportion of more than 40, 50 or 60% of the total signal are used. This ensures that only image points that represent the introduced object are depicted in the calculated localization image.

The localization image can also be generate by automatically determining a slice position for the acquisition of additional MR images in order to place the slice plane such that the introduced object is visible in the additionally acquired MR images, in addition to the surrounding tissue. For example, the introduced object could be depicted in color in the additional MR images or in the localization image.

Another possibility to calculate the localization image is to add the first and second MR signals, or to subtract them from one another. A first MR image data set can hereby be created in which essentially only the introduced object is shown, and a second MR image data set can be created in which essentially only the predominant tissue is shown. The proportion of the introduced object that is shown in the localization image relative to the total tissue can then be calculated with the aid of the two MR data sets. For example, this is possible by dividing the signal intensity in the first image data set by the added signal intensities of the first and second MR image data sets.

The introduced object advantageously consists essentially of silicone. Silicone is a biocompatible material and exhibits a chemical shift of approximately 4.7 ppm (parts per million) relative to water. Furthermore, no additional signal is present in the MR spectrum at the same chemical shift in human tissue. However, other materials can also be used that are biocompatible and have a chemical shift different than that of water.

The invention furthermore concerns a magnetic resonance system which is designed to depict this object introduced into the examination region, wherein the MR system has an MR image data acquisition unit to acquire the first and second MR signals as described above. Furthermore, a computer is provided that calculates the localization image with the use of the first and second MR signals. A display unit is likewise provided to display the calculated localization image.

### BRIEF DESCRIPTION OF THE DRAWINGS

FIG. 1 schematically illustrates an MR system with which an object introduced into an examination region can be depicted.

FIG. 2 shows an example of the different chemical shift between water and silicone.

FIG. 3 is a section from an imaging sequence with which first and second MR signals can be acquired to calculate a localization image.

FIG. 4 is a flowchart that contains the basic steps to calculate the localization image in accordance with the invention.

### DESCRIPTION OF THE PREFERRED EMBODIMENTS

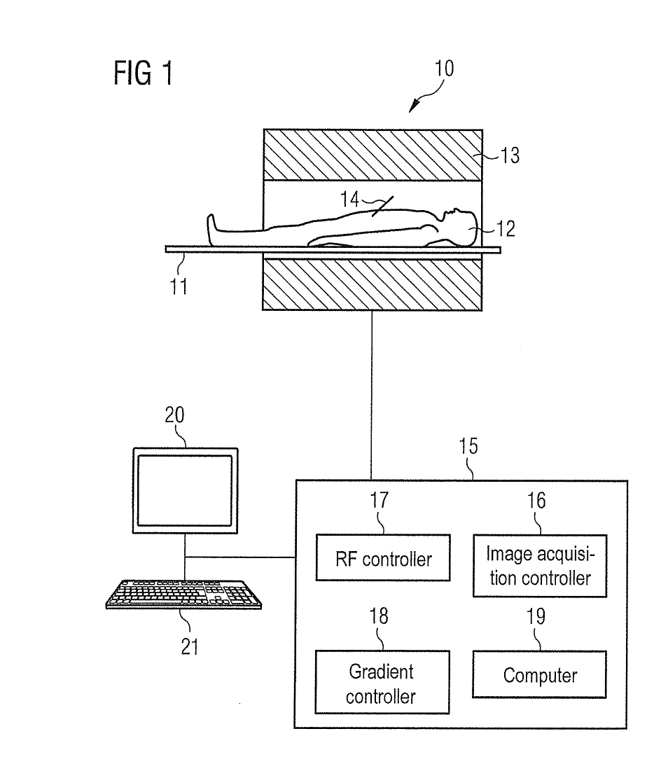

FIG. 1 schematically shows a magnetic resonance (MR) system 10 in which the examination subject 12 arranged on a bed 11 is driven into a magnet unit 13 in order to be able to acquire MR images of the examination subject 12. In the shown example, an interventional application simultaneously takes place in which an object 14—a catheter or a biopsy needle, for example—is introduced into the examination subject. The MR system has a central controller 15. This central controller has an image acquisition controller 16 that controls the radiation of radio-frequency pulses and switching of magnetic field gradients via an RF controller 17 and a gradient controller 18 such that MR signals are acquired with coils (not shown) from a desired examination region. The details of how MR images of an examination region can be generated from MR data acquired by radiation of radio-frequency pulses and switching of magnetic field gradients are known those skilled in the art and thus need not be explained in further detail herein.

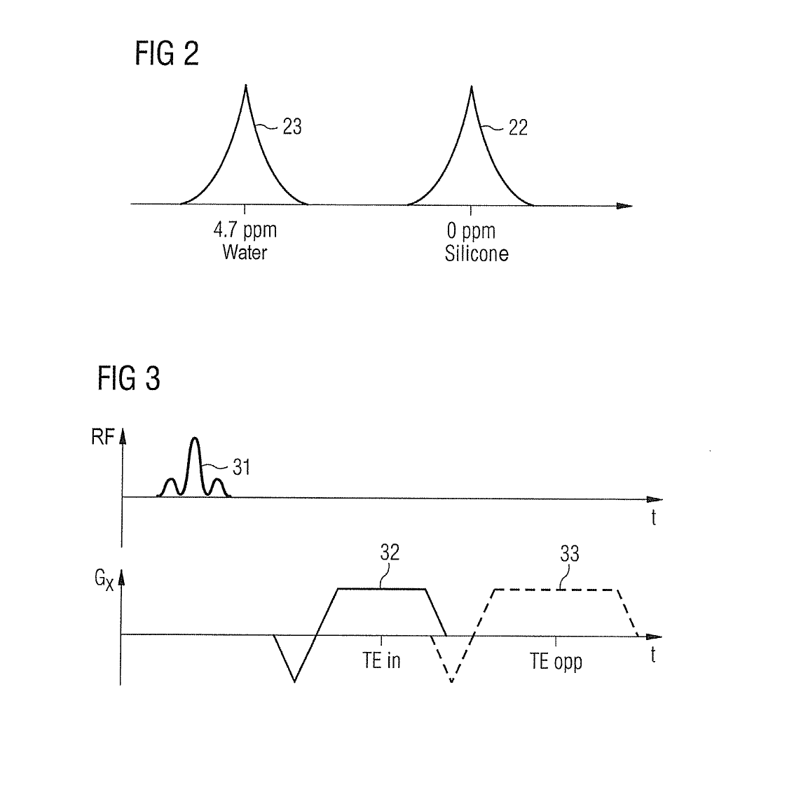

In interventional applications, for the treating physician it is important that he or she receives information about the position of the introduced object in the examination region. The introduced object can consist of silicone, for example, or have a silicone case. The goal is now to depict the introduced object in the MR images. Since the introduced objects normally have a very small spatial extent, it is not simple to detect the introduced object in the acquired MR images. The manner by which this is made possible according to the invention is explained in detail in the following with reference to FIGS. 2-4.

As is apparent (among other things) from FIG. 2, silicone has a different chemical shift than water. The water protons in the human body typically exhibit a chemical shift of 4.7 ppm relative to a reference material, as is apparent from the water spectrum 23. In contrast to this, the silicone spectrum 22 has a chemical shift of 0 ppm relative to the reference material (not visible in the MR image). This different chemical shift means a different resonance frequency of the associated nuclei in the magnetic field, and thus a different phase position of the respective magnetization at the echo point in time.

One possibility to depict the image points that contain silicone separate from the image points that contain water is to acquire first MR signals once in which the two components (water and silicone) have the same phase position, whereas they have an opposite phase position at a second acquisition. This method (known from Dixon) to separate fat and water can be used in the present case for separate depiction of the silicone.

As is partially shown in FIG. 3, for this a gradient echo sequence can be used in which a first MR signal is acquired at a first echo time TE1 and the second MR signal is acquired at a second echo time TE2. After radiating an RF pulse 31 of a gradient echo sequence, the readout gradient can be switched after switching the slice selection and phase coding gradients (not shown) such that both components—the introduced object and the surrounding tissue, for example water and silicone or fat and silicone—have the same phase position at the first echo point in time TEin, whereas they have the opposite phase position at an additional echo TEopp. The signal echoes are generated with gradient echoes since, in this manner of echo generation, the different shift affects the acquired signal. The bipolar gradient circuits 32 and 33 are respectively used to read out the gradient echoes. The connection between the echo times and the frequency differences due to the different chemical shift is as follows:

$$TEin = \frac{1}{fcs} \qquad ( 1 )$$

wherein TEin corresponds to the first MR signal in which both tissues are in phase. Here fcs is the frequency difference due to the chemical shift that depends on (among other things) the B₀ field strength. As is apparent in connection with FIG. 2, the frequency difference between water and silicone amounts to 4.7 ppm, which corresponds to approximately 300 Hz at a magnetic field strength of 1.5 Tesla.

The echo time for the opposite phase position is:

$$TEopp = \frac{1}{2 fcs} \qquad ( 2 )$$

It follows from this that TEin=3.33 ms. The echo with the opposite phase position lies at TEopp=1.66 ms ( 1/600). The next echo with parallel phase position would then be at 4.99 ms etc. A localization image in first approximation can be calculated as follows from the two MR signals that are acquired at the echo points in time TEin and TEopp. Depending on the speed of the gradient circuits, the parallel or opposite phase position is acquired first. If the T2 decay times are not taken into account, the signal at the echo point in time TEin is composed as follows:

I₀=I_{W}+I_{S}, (3)

wherein I₀ is the total signal, I_{W} is the aqueous portion and I_{S} is the silicone portion. At the second echo point in time the signal is as follows:

l₁=l_{W}−I_{S}, (4)

since here the magnetization of the silicone is opposite the magnetization of the water. An MR image data set I_{W} that essentially depicts only the predominant tissue and an MR image data set I_{S} that essentially depicts only the introduced object can be calculated from this:

$$\begin{matrix}{I}_{W} = \frac{( {I}_{0} + {I}_{1} )}{2} \\ and & ( 5 ) \\ {I}_{S} = \frac{( {I}_{0} - {I}_{1} )}{2} . & ( 6 )\end{matrix}$$

From this a localization image can be calculated in which the signal intensity is proportional to the proportion of the introduced silicone:

$${I}_{S} = \frac{{I}_{S}}{( {I}_{W} + {I}_{S} )} . \qquad ( 7 )$$

Referring again to FIG. 1, this localization image can be calculated in the computer 19 and presented on a display unit 20. Furthermore, an input unit 21 is provided with which the MR system 10 can be controlled. The localization image calculated with the above Equation (8)—in which the signal intensity in each image point is proportional to the proportion of the silicone in the total signal—can subsequently be presented. Essentially only the introduced object is then shown in this localization image. To improve the presentation, a limit value of the intensity can furthermore be determined so that (for example) only intensities at which the silicone proportion or, respectively, the proportion of the introduced object in the total signal is greater than a predetermined limit value are shown.

Furthermore, the localization image can then be used in order to automatically implement the slice determination for additional MR acquisitions, wherein this slice determination takes place such that the introduced object is shown together with the examination region around the object in the acquired slice. The calculated localization image could likewise be superimposed with other MR images that show the surrounding tissue in order to better present the position of the introduced object in the examination region.

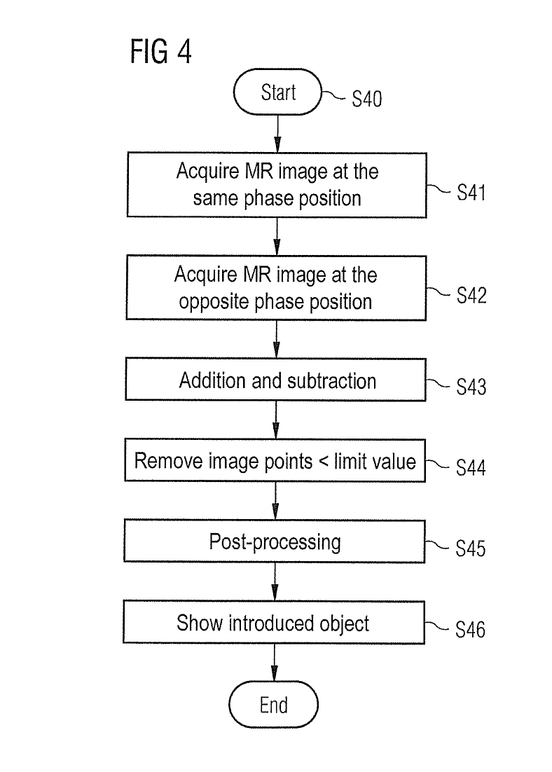

A summary of the steps to generate the localization image takes place in connection with FIG. 4. After the start of the method in Step S40, in Step S41 a first MR image is acquired in which the different tissue—i.e. the introduced object and the tissue present in the examination region—have the same phase position (Step S41). Furthermore, in Step S42 a second MR image is acquired in which the two components have an essentially opposite phase position. The order of Steps S41 and S42 can also be exchanged. This second MR image can be acquired in a second, separate imaging sequence with the echo time TEopp; however, the acquisition of the two MR signals is also possible in a double echo imaging sequence. As mentioned above, in Step S43 the acquired MR signals are added and subtracted to calculate the intensities I₀ and I₁. In a further step S44, if necessary the image points with a signal intensity value smaller than a limit value can be removed in the localization image calculated from said intensities according to Equation (7). In an optional Step S45 (designated as post-processing), for example, the introduced object can be emphasized in color in the localization image. In Step S46, the introduced object can finally be presented in an MR image, either alone or superimposed with other MR images.

Furthermore, in one step (not shown) the calculated position of the introduced object can be passed to the image acquisition controller, which then automatically places slice planes for additional MR acquisitions so that the introduced object is visible in the MR image then created. One possibility to superimpose the localization image is the superimposition with a phase image of the examination region.

The imaging sequence to acquire the first and second MR signals at the echo point in time TEin and TEopp can be a 2D or 3D imaging sequence. Although the present invention was described in connection with silicone, other materials are conceivable that have a given chemical shift relative to the water protons.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to show, in a magnetic resonance image of a subject, an object introduced into an examination region of the subject, said object having a known chemical shift relative to tissue that is predominant in said examination region, said method comprising:
acquiring magnetic resonance signals from the subject, with the object introduced into the subject, with a magnetic resonance data acquisition system; and
providing said magnetic resonance signals to a processor and, in said processor, using said respectively different chemical shifts of the introduced object and of the predominant tissue to calculate, from the acquired magnetic resonance signals, a localization image in which substantially only the introduced object is shown.

2. A method as claimed in claim 1 wherein the step of acquiring said magnetic resonance signals comprises:
acquiring first magnetic resonance signals by executing a gradient echo imaging sequence in said magnetic resonance data acquisition system that causes a magnetization of the predominant tissue in the examination region, and a magnetization of the introduced object, to have substantially a same phase position at an echo point in time of said gradient echo imaging sequence;
acquiring second magnetic resonance signals by implementing a gradient echo imaging sequence with said magnetic resonance data acquisition system that causes a magnetization of the predominant tissue in the examination region, and a magnetization of the introduced object, to have substantially opposite phase positions at said echo point in time of said gradient echo imaging sequence;
and wherein the step of calculating said localization image comprises using said first and second MR signals to calculate said localization image in which a signal intensity corresponds to a proportion of the introduced object in a totality of said first and second magnetic resonance signals;
and wherein said method further comprises displaying said localization image.

3. A method as claimed in claim 2 comprising calculating said localization image by adding said first and second MR signals to generate a first MR image data set in which substantially only the introduced object is shown, and subtracting said first and second MR signals from each other to generate a second MR image data set in which substantially only predominant tissue is shown, and calculating a proportion of the introduced object in the localization image relative to a totality of tissue using said first and second magnetic resonance data sets.

4. A method as claimed in claim 1 comprising calculating said localization image using only image points at which a signal proportion of the introduced object in a totality of the magnetic resonance signals is greater than a predetermined limit value.

5. A method as claimed in claim 1 comprising showing the introduced object, which is localized with said localization image, in an additional magnetic resonance image acquired by said magnetic resonance data acquisition system.

6. A method as claimed in claim 1 comprising in said processor, generating, from said localization image, a designation of a slice position of the subject for use by said magnetic resonance data acquisition system in acquiring additional magnetic resonance images of the subject in which the introduced object is shown.

7. A method as claimed in claim 1 wherein said introduced object is comprised of silicone.

8. A method as claimed in claim 1 comprising, in said processor, automatically determining a position of said introduced object in said examination region.

9. A magnetic resonance system to show, in a magnetic resonance image of a subject, an object introduced into an examination region of the subject, said object having a known chemical shift relative to tissue that is predominant in said examination region, said magnetic resonance system comprising:
a magnetic resonance data acquisition unit;
a control unit configured to operate the magnetic resonance data acquisition unit to acquire magnetic resonance signals from the subject, with the object introduced into the subject, in the magnetic resonance data acquisition unit; and
a processor provided with said magnetic resonance signals, said processor being configured to use said respectively different chemical shifts of the introduced object and of the predominant tissue to calculate, from the acquired magnetic resonance signals, a localization image in which substantially only the introduced object is shown.
